(19) United States
(12) Patent Application Publication
(10) Pub. No.: US 2026/0219260 A1
(43) Pub. Date: Jul. 30, 2026

(54) BIOASSAY METHOD FOR HANGESHASHINTO USING PHARMACOLOGICAL ACTIVITY AS INDICATOR

(71) Applicant: TSUMURA & CO., Tokyo (JP)

(72) Inventor: Ryota IMAI, Inashiki-gun (JP)

(73) Assignee: TSUMURA & CO., Tokyo (JP)

(21) Appl. No.: 19/448,346

(22) Filed: Jan. 14, 2026

(30) Foreign Application Priority Data
Jan. 27, 2025 (JP) 2025-011644

(51) Int. Cl. G01N 33/50; C12N 5/071

(52) U.S. Cl. CPC G01N 33/5044; C12N 5/0629; C12N 2503/02; G01N 2405/00

(57) ABSTRACT

A bioassay method for evaluating pharmacological activity which can be used for quality control of hangeshashinto is provided. A bioassay method for pharmacological activity of hangeshashinto characterized by adding an inflammatory cytokine a and hangeshashinto-containing test sample to a human oral keratinocyte, culturing the human oral keratinocyte, and then measuring the prostaglandin E2 concentration of the culture supernatant, and a quality control method of hangeshashinto using the bioassay method.

### BACKGROUND OF THE INVENTION

### 1. Field of the Invention

The present invention relates to a bioassay method for pharmacological activity of hangeshashinto.

### 2. Description of Related Art

A kampo preparation is a pharmaceutical prepared by blending crude drugs, and not all the active ingredients thereof are specifically identified. Furthermore, the kampo preparation does not exert the effect through a single active ingredient alone, but rather acts through combined action of the ingredients. Accordingly, for securing the quality of a kampo preparation, a method capable of evaluating the kampo preparation as a whole is ideal.

Methods for evaluating the quality of a kampo preparation include a method of measuring the individual ingredients and then comprehensively evaluating, and a bioassay for evaluating pharmacological activity using a biological material.

The present applicant has reported a method of evaluating the amount of released serotonin in cultured serotonin-producing cells as a bioassay method for evaluating pharmacological activity which can be used for quality control of daikenchuto (PTL 1). Moreover, the applicant has reported evaluation methods as patents for bioassay methods for evaluating pharmacological activity which can be used for quality control of yokukansan (PTLs 2 and 3).

However, it is required to find a bioassay method suitable for each kampo preparation because the bioassay method for daikenchuto cannot be applied to a kampo preparation having different pharmacological activity, and for example, no bioassay method for evaluating pharmacological activity which can be used for quality control of hangeshashinto, which has different pharmacological activity from that of daikenchuto, has been reported so far.

### CITATION LIST

### Patent Literature

PTL 1: WO2012/073881
PTL 2: WO2009/008266
PTL 3: WO2009/101700

### SUMMARY OF THE INVENTION

Therefore, a problem to be solved by the invention is to provide a bioassay method for evaluating pharmacological activity which can be used for quality control of hangeshashinto.

As a result of intensive studies to solve the problem, the present inventors have found that pharmacological activity which can be used for quality control of hangeshashinto can be evaluated using, as an indicator, the concentration of prostaglandin E2 at which human oral keratinocytes release through stimulation with an inflammatory cytokine, and the invention has been thus completed.

That is, the invention is as follows.
[1] A bioassay method for pharmacological activity of hangeshashinto characterized by adding an inflammatory cytokine and a hangeshashinto-containing test sample to a human oral keratinocyte, culturing the human oral keratinocyte, and then measuring the prostaglandin E2 concentration of the culture supernatant.
[2] The bioassay method for pharmacological activity of hangeshashinto according to [1] in which the inflammatory cytokine is interleukin-1β.
[3] The bioassay method for pharmacological activity of hangeshashinto according to [1] or [2] in which a nonsteroidal anti-inflammatory agent is further used as a positive control.
[4] The bioassay method for pharmacological activity of hangeshashinto according to [3] in which the nonsteroidal anti-inflammatory agent is indomethacin or diclofenac sodium.
[5] The bioassay method for pharmacological activity of hangeshashinto according to any one of [1] to [4] in which the culture is conducted for four to 36 hours.
[6] A quality control method of hangeshashinto characterized by including the following steps (a), (b), and (d):
(a) a step of adding an inflammatory cytokine to a human oral keratinocyte, culturing the human oral keratinocyte, and then measuring the prostaglandin E2 concentration of the culture supernatant;
(b) a step of adding the inflammatory cytokine and a hangeshashinto-containing test sample to a human oral keratinocyte, culturing the human oral keratinocyte, and then measuring the prostaglandin E2 concentration of the culture supernatant; and
(d) a step of comparing the prostaglandin E2 concentrations measured in the steps (a) and (b).
[7] A quality control method of hangeshashinto characterized by including the following steps (a) to (c) and (e):
(a) a step of adding an inflammatory cytokine to a human oral keratinocyte, culturing the human oral keratinocyte, and then measuring the prostaglandin E2 concentration of the culture supernatant;
(b) a step of adding the inflammatory cytokine and a hangeshashinto-containing test sample to a human oral keratinocyte, culturing the human oral keratinocyte, and then measuring the prostaglandin E2 concentration of the culture supernatant;
(c) a step of adding the inflammatory cytokine and a nonsteroidal anti-inflammatory agent as a positive control to a human oral keratinocyte, culturing the human oral keratinocyte, and then measuring the prostaglandin E2 concentration of the culture supernatant; and
(e) a step of comparing the prostaglandin E2 concentrations measured in the steps (a) to (c).
[8] The quality control method of hangeshashinto according to [6] or [7] in which the inflammatory cytokine is interleukin-1β.
[9] The quality control method of hangeshashinto according to [7] or [8] in which the nonsteroidal anti-inflammatory agent is indomethacin or diclofenac sodium.
[10] The quality control method of hangeshashinto according to any one of [6] to [9] in which the culture is conducted for four to 36 hours.
[11] The quality control method of hangeshashinto according to any one of [7] to in which the prostaglandin E2 concentration measured in the step (c) is compared with the prostaglandin E2 concentration of another lot to correct the values of lots.

In the bioassay method for pharmacological 1 activity of hangeshashinto of the invention, the pharmacological activity of hangeshashinto can be assayed with excellent reproducibility using, as an indicator, the concentration of prostaglandin E2 at which human oral keratinocytes release through stimulation with an inflammatory cytokine.

Moreover, in the quality control method of hangeshashinto of the invention, the quality of hangeshashinto can be controlled with excellent reproducibility through assay of the pharmacological activity of hangeshashinto using, as an indicator, the concentration of prostaglandin E2 at which human oral keratinocytes release through stimulation with an inflammatory cytokine.

### BRIEF DESCRIPTION OF THE DRAWINGS

FIG. 1 shows the PGE2 concentrations in the HOK cells with the respective cell densities which were treated with IL-1β for 2 h in Reference Example 1 (n=3 wells, error bar=SEM).

FIG. 2 shows the PGE2 concentrations in the HOK cells with the respective cell densities which were treated with IL-1β for 6 h in Reference Example 1 (n=3 wells, error bar=SEM).

FIG. 3 shows the PGE2 concentrations in the HOK cells with the respective cell densities which were treated with IL-1β for 24 h in Reference Example 1 (n=3 wells, error bar=SEM).

FIG. 4 shows the PGE2 concentrations in the HOK cells with the respective cell densities which were treated with IL-1β and/or indomethacin for 6 h in Reference Example 1 (n=3 wells, error bar=SEM).

FIG. 5 shows the PGE2 concentrations in the HOK cells with the respective cell densities which were treated with IL-1β and/or indomethacin for 24 h in Reference Example 1 (n=3 wells, error bar=SEM).

FIG. 6 shows the PGE2 concentrations in the HOK cells with the respective cell densities which were treated with IL-1β and/or indomethacin for 6 h or 24 h in Reference Example 1 (n=3 wells, error bar=SEM).

FIG. 7 shows the influence on PGE2 production in the HOK cells with a treatment period of 24 h in Example 1 (n=3 wells, error bar=SEM).

FIG. 8 shows the influence on PGE2 production in the HOK cells with treatment with hangeshashinto alone (24 h) in Example 1 (n=3 wells, error bar=SEM).

FIG. 9 shows the PGE2 inhibition ratios through treatment with the representative hangeshashinto extract lot (30 μg/mL) in the HOK cells on the respective experiment dates in Example 2 (n=3 or 4 wells, error bar=SD). In the figure, each column shows an experiment date, and each sub-number shows a plate number of the same date.

FIG. 10 shows the PGE2 inhibition ratios through treatment with the respective lots of hangeshashinto extract (30 μg/mL) in the HOK cells in Example 2 (n=3 or 4 wells, IL-1β and rep.: n=44 wells, One-way ANOVA followed by Dunnett's test, *** p<0.001, error bar=SEM).

### DESCRIPTION OF EMBODIMENTS

The bioassay method for pharmacological activity of hangeshashinto of the invention (hereinafter, referred to “the method of the invention”) is a method characterized by adding an inflammatory cytokine and a hangeshashinto-containing test sample to a human oral keratinocyte, culturing the human oral keratinocyte, and then measuring the prostaglandin E2 concentration of the culture supernatant.

An example of the hangeshashinto used as the subject of bioassay in the method of the invention is a pulverized extract of a mixed crude drug having the composition in Table 1. Another example thereof is a preparation formed into granules or the like by adding a component which is approved as a pharmaceutical additive. An example of the hangeshashinto is Tsumura-Kampo hangeshashinto extract granules (for medical use) or the like.

TABLE 1

| Name | Amount |
|---|---|
| JP Pinellia Tuber | 5.0 g |
| JP Scutellaria Root | 2.5 g |
| JP Processed Ginger | 2.5 g |
| JP Glycyrrhiza | 2.5 g |
| JP Jujube | 2.5 g |
| JP Ginseng | 2.5 g |
| JP Coptis Rhizome | 1.0 g |

Examples of the component which is approved as a pharmaceutical additive include an excipient such as starch, lactose, sucrose, mannitol, carboxymethyl cellulose, cornstarch, and an inorganic salt, a disintegrant, a surfactant, a lubricant, a fluidizing agent, a flavoring agent, a colorant, and an aroma.

The human oral keratinocytes (HOKs) used in the method of the invention are not particularly limited, and for example, a commercial product such as human oral keratinocytes (ScienCell Research Laboratories, Cat No. 2610) can be used.

An inflammatory cytokine and a hangeshashinto-containing test sample are added to the human oral keratinocytes. The timings for adding the inflammatory cytokine and the hangeshashinto-containing test sample are not particularly limited and may be simultaneous. Alternatively, the inflammatory cytokine may be added first, or the hangeshashinto-containing test sample may be added first. However, it is preferable to add the inflammatory cytokine and then add the hangeshashinto-containing test sample. The interval of the addition is not particularly limited, and is, for example, two to 10 minutes and preferably four to six minutes.

When the inflammatory cytokine is added to the human oral keratinocytes, prostaglandin E2 is produced from the human oral keratinocytes. The inflammatory cytokine is not particularly limited, and examples thereof include interleukin-1β, TNF-α (Tumor Necrosis Factor-α), interleukin-6, interleukin-8, IFN-γ (interferon-gamma), and MCP-1. Here, in the invention, in addition to the above inflammatory cytokines, for example, a substance which evokes inflammation such as polysaccharides including LPSs (lipopolysaccharides) can also be similarly used. Of these inflammatory cytokines, interleukin-1β is preferable. Those which are commercially available as a pharmaceutical, a reagent or the like thereof can be used.

The amount of the inflammatory cytokine added to the human oral keratinocytes is not particularly limited, and is, for example, 0.5 to 40 ng/ml, preferably 1 to 15 ng/ml and more preferably 1 ng/ml based on 2 to 4×10⁴ cells/well of the human oral keratinocytes.

The hangeshashinto-containing test sample added to the human oral keratinocytes refers to hangeshashinto or a kampo preparation which contains the same kind of crude drugs as those of hangeshashinto and which further contains another component.

The amount of the hangeshashinto-containing test sample added to the human oral keratinocytes is not particularly limited, and is, for example, 5 to 500 μg/mL, preferably 10 to 300 μg/mL and more preferably 30 μg/mL in terms of the soluble solid content of the hangeshashinto based on 2 to 4×10⁴ cells/well of the human oral keratinocytes.

Here, the concentration of the hangeshashinto-containing test sample may be adjusted, for example, by diluting the hangeshashinto-containing test sample with a culture medium for human oral keratinocytes such as Oral Keratinocyte Medium (OKM, Cat. #2611) which contains FBS (fetal bovine serum) as serum or which does not contain FBS, a general physiological buffer such as PBS, or an organic solvent such as DMSO.

After the inflammatory cytokine and the hangeshashinto-containing test sample are added to the human oral keratinocytes, the human oral keratinocytes are cultured. The culture condition is, for example, the condition described in the manual attached to human oral keratinocytes (HOKs) derived from healthy human oral mucosa (ScienCell Research Laboratories, Cat No. 2610) or the like. Specifically, examples of the culture medium in which the human oral keratinocytes can be cultured include Oral Keratinocyte Medium (OKM, Cat. #2611) which contains FBS (fetal bovine serum) as serum or which does not contain FBS. Moreover, the culture period for culturing the human oral keratinocytes is not particularly limited, and is, for example, four to 36 hours, preferably six to 24 hours and more preferably 24 hours, because accurate assay is not possible when the culture period is too short. The culture temperature is not particularly limited as long as the human oral keratinocytes can be cultured at the temperature, and the culture temperature is, for example, 36 to 38° C. and preferably 37° C. Moreover, the culture is preferably conducted in the presence of carbon dioxide and more preferably in the presence of 5% carbon dioxide. Furthermore, the culture is preferably static culture.

After the culture, the culture supernatant is collected according to a normal method, and the prostaglandin E2 concentration of the culture supernatant is measured. The method for measuring the prostaglandin E2 concentration of the culture supernatant is not particularly limited, and for example, a commercial prostaglandin E2 assay kit such as ELISA Kit (Cat #514010) manufactured by Cayman's chemical can be used.

By measuring the prostaglandin E2 concentration of the culture supernatant by the method of the invention in the above manner, the pharmacological activity of hangeshashinto can be biologically assayed.

In the method of the invention, a nonsteroidal anti-inflammatory agent is preferably further used as a positive control. Through correction using the measurement value of the positive control as a basis, the accuracy of the assay can be checked, or the assay results of a plurality of test sample lots can be compared. The correction and the comparison can be made by methods known to one skilled in the art.

The nonsteroidal anti-inflammatory agent used as the positive control is not particularly limited, and examples thereof include indomethacin, diclofenac sodium, loxoprofen sodium, acetylsalicylic acid, and celecoxib, naproxen. Of the nonsteroidal anti-inflammatory agents, indomethacin or diclofenac sodium is preferable.

The nonsteroidal anti-inflammatory agent is added to the human oral keratinocytes instead of the hangeshashinto-containing test sample, and the amount to be added is not particularly limited, and is, for example, 0.05 to 100 μM, preferably 0.1 to 1 μM and more preferably 1 μM based on 2 to 4×10⁴ cells/well of the human oral keratinocytes. Moreover, the timing for adding the nonsteroidal anti-inflammatory agent may be the same as the timing of the case using the hangeshashinto-containing test sample.

Here, the concentration of the nonsteroidal anti-inflammatory agent may be adjusted, for example, by diluting with an organic solvent such as DMSO or the like.

When the quality of hangeshashinto as a product is evaluated by the method of the invention, the pharmacological activity values (the prostaglandin E2 concentrations of culture supernatants) of a plurality of lots of hangeshashinto which has been clinically found to have a pharmacological effect are measured, and a standard range is set based on the values. Next, by the same method, the pharmacological activity value of a quality test sample of hangeshashinto to be evaluated is measured. Then, the equivalence is evaluated based on whether the pharmacological activity value of the sample is in the set standard range, and the sample which is found to be equivalent is determined as an acceptable product. The quality control may be conducted in this manner.

A preferable embodiment of the quality control method using the method of the invention is as follows.

A quality control method of hangeshashinto characterized by including the following steps (a), (b), and (d):
(a) a step of adding an inflammatory cytokine to a human oral keratinocyte, culturing the human oral keratinocyte, and then measuring the prostaglandin E2 concentration of the culture supernatant;
(b) a step of adding the inflammatory cytokine and a hangeshashinto-containing test sample to a human oral keratinocyte, culturing the human oral keratinocyte, and then measuring the prostaglandin E2 concentration of the culture supernatant; and
(d) a step of comparing the prostaglandin E2 concentrations measured in the steps (a) and (b).

Another preferable embodiment of the quality control method using the method of the invention is as follows.

A quality control method of hangeshashinto characterized by including the following steps (a) to (c) and (e):
(a) a step of adding an inflammatory cytokine to a human oral keratinocyte, culturing the human oral keratinocyte, and then measuring the prostaglandin E2 concentration of the culture supernatant;
(b) a step of adding the inflammatory cytokine and a hangeshashinto-containing test sample to a human oral keratinocyte, culturing the human oral keratinocyte, and then measuring the prostaglandin E2 concentration of the culture supernatant;
(c) a step of adding the inflammatory cytokine and a nonsteroidal anti-inflammatory agent as a positive control to a human oral keratinocyte, culturing the human oral keratinocyte, and then measuring the prostaglandin E2 concentration of the culture supernatant; and
(e) a step of comparing the prostaglandin E2 concentrations measured in the steps (a) to (c).

Hereinafter, the invention will be explained specifically referring to Examples of the invention, but the invention is not limited to the Examples.

### Reference Example 1

### Preliminary Test for Constructing Bioassay System:

### <Examination of Conditions of Cell Count, IL-1β Concentration, and Treatment Period>

### (1) Culture Method

Human oral keratinocytes (HOKs) derived from healthy human oral mucosa (ScienCell Research Laboratories, Cat No. 2610) which were stored at −80° C. were thawed, and then the cell suspension (1 mL) was quickly added to 10% FBS culture medium (9 mL). After sufficiently suspending, the suspension was centrifuged (4° C., 1000 rpm (=190 g), 5 min; TOMY, AX-511), and the supernatant was removed. An adequate amount of 10% FBS culture medium was added to the cell pellets, and the cells were seeded on a 60-mm dish coated with poly-L-lysine (5 mL/dish; IWAKI, Cat #4010-040) at 0.625, 1.25 or 2.5×10⁶ cells/dish or the like or on a 100-mm dish coated with poly-L-lysine (10 mL/dish; IWAKI, Cat #4020-040) at 0.5, 0.75 or 1.0×10⁶ cells/dish or the like. On the next day, the cells were washed with PBS, and the culture medium was changed (the culture medium was not always changed depending on the cell condition). The cells with about 60 to 100% confluency were detached three or four days after seeding the cells, suspended in 2% FBS culture medium and seeded on a 96-well plate coated with poly-L-lysine (Sumitomo Bakelite Co., Ltd., Cat #MS-0096L) at 0.5, 1.0, 2.0 or 4×10⁴ cells/well or the like (100 μL/well). The cells after culturing overnight were used for the assay.

### (2) PGE2 Measurement

The supernatants of the cells cultured overnight in the 96-well plate coated with poly-L-lysine were removed, and the cells were washed with PBS. After washing, IL-1β (PeproTech, Cat No.: 200-01B) was added (100 μL/well), and (when a test sample (or a positive control) was used) a test sample was added (100 μL/well) soon thereafter (about five minutes). The final volume for the assay was 200 μL/well. IL-18 and the test samples were each produced at 2× concentration and adjusted in such a manner that the concentration would become the aimed concentration after being mixed in the well. After leaving still in the presence of 5% CO₂ at 37° C. for the aimed culture period, the plate was centrifuged (25° C., 1000 rpm (=220 g), 1 min; TOMY, EX-135), and then 100 μL of the culture supernatants were collected and used as samples. The samples were either used immediately or stored at −80° C. until the measurement. The cells after collecting the supernatants were used for measuring the cytotoxicity. The PGE2 in the supernatants was measured using ELISA Kit (Cat #514010) manufactured by Cayman's chemical. The basic operational method was in accordance with the protocol of the manufacturer. The samples were diluted at 10-fold with ELISA buffer and used. After incubating at room temperature for 45, 60 or 75 minutes, the absorbances at 405 nm were measured using a plate reader (Thermo Fisher Scientific, Multiskan GO), and appropriate measurement results were used following the protocol of the manufacturer.

### (3) Measurement of Cytotoxicity

The cytotoxicity of collecting the cells after collecting the supernatants was measured using Cell Counting Kit-8 (CCK-8; DOJINDO LABORATORIES, Cat #CK04). The basic operational method was in accordance with the protocol of the manufacturer. After the cells were washed with 200 μL of PBS, a 10% CCK-8 solution which was diluted with a serum-free culture medium warmed to 37° C. in advance was added at 100 μL/well. Then, the cells were cultured in a CO₂ incubator. After culturing for two hours, the absorbances at 450 nm were measured using a plate reader (Thermo Fisher Scientific, Multiskan GO), and the cytotoxicity was thus measured.

### (4) Statistical Processing

The results are shown by average value±standard deviation (SD) or average value±standard error (SEM). For the significance between groups, a significance of 5% or less was regarded as the significance level. Prism 7 was used as the statistical analysis software.

### (5) Results

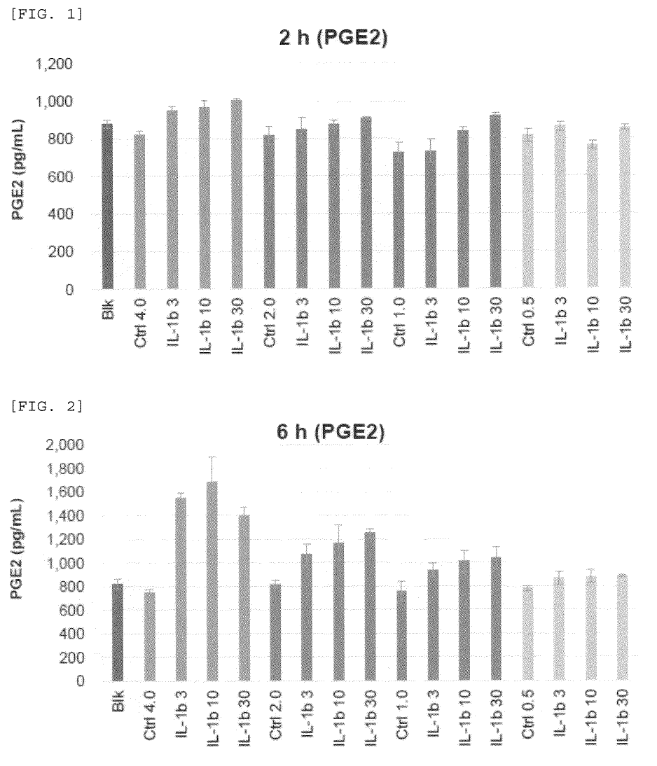

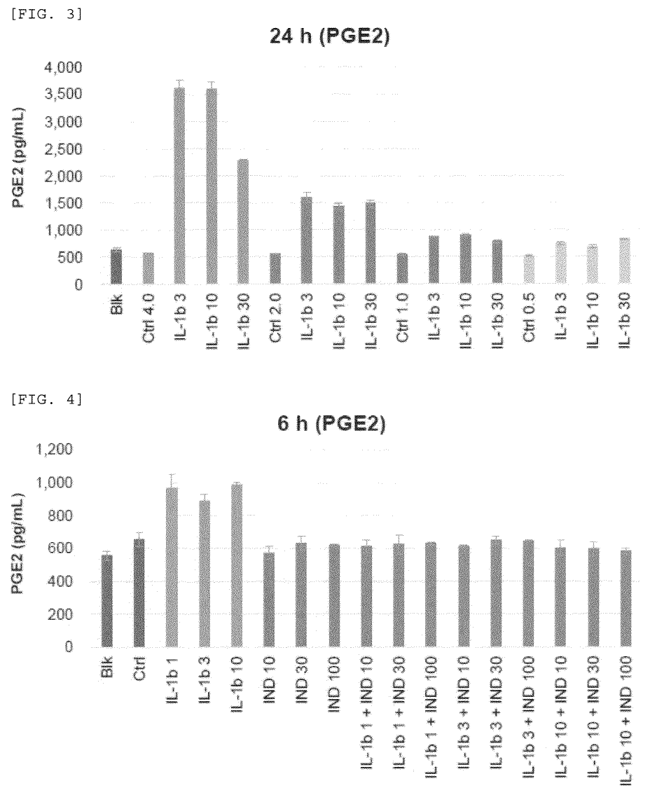

The conditions set were the cell counts of 0.5, 1, 2, and 4×10⁴ cells/well, IL-1β at 3, 10, and 30 ng/ml, and the treatment periods of 2, 6, and 24 h. Under the conditions, the PGE2 values in the supernatants were measured using the ELISA kit. With the treatment period of 2 h, PGE2 was not produced under any condition. On the other hand, with the treatment periods of 6 and 24 h, there was a tendency towards production of PGE2 in response to the cell count although no response to the concentration of IL-1β was observed (FIGS. 1 to 3). Moreover, the influence on the cytotoxicity under the conditions with the treatment periods of 2, 6, and 24 h was examined using CCK-8. When the cytotoxicity values were calculated using the value of the control group with each cell count regarded as 100%, no remarkable influence was observed although a slight change was found under some conditions (not shown in the figures).

From the results, it was determined that the conditions with a cell count of 4×10⁴ cells/well and treatment periods of 6 and 24 h, under which PGE2 was produced due to IL-1β, were suitable.

### Examination 1 of Conditions of IL-1β and Indomethacin Concentrations

Regarding the inhibitory action on the increase in PGE2 caused by IL-1β, the reactivity of indomethacin (a positive control having a PGE2 inhibitory action: test sample: FUJIFILM Wako Pure Chemical Corporation, Cat No.: 093-02473) was examined in the same manner as in <Examination of Conditions of Cell Count, IL-1β Concentration, and Treatment Period> above. Indomethacin was dissolved in DMSO at a concentration of 100 mM, and the solution was stored at −80° C. as a stock solution and used after appropriate dilution. The examination was conducted under the conditions of the cell count fixed at 4×10⁴ cells/well, IL-1β at 1, 3, and 10 ng/ml, indomethacin at 10, 30, and 100 μM, and the treatment periods of 2, 6, and 24 h.

With the treatment period of 2 h, PGE2 production was not caused by IL-1β (1, 3, and 10 ng/ml) (not shown in the figures). With the treatment period of 6 h, PGE2 was produced due to IL-1β (1, 3, and 10 ng/ML) (FIG. 4). With the treatment period of 24 h, PGE2 was also produced due to IL-1β (1, 3, and 10 ng/ml), but no response to the concentration was observed (FIG. 5). The PGE2 production caused by IL-13 (3 and 10 ng/ml) with all the treatment periods was the same as that in <Examination of Conditions of Cell Count, IL-1β Concentration, and Treatment Period> above, and the results were reproducible. Moreover, indomethacin at all the concentrations (10, 30, and 100 μM) with the treatment periods of 6 and 24 h inhibited the PGE2 production caused by IL-1β (1, 3, and 10 ng/ml) (FIGS. 4 and 5). PGE2 was not changed by indomethacin alone (10, 30, and 100 μM) with all the treatment periods. Here, no remarkable cytotoxicity was observed under any of the conditions (not shown in the figures).

From the results, because no response to the concentration was observed with IL-1β at 1, 3, and 10 ng/ml, it was determined that examination at lower concentrations was necessary. Moreover, it was supposed that indomethacin would exhibit an inhibitory action on PGE2 production at a concentration of 10 μM or less.

### Examination 2 of Conditions of IL-1β and Indomethacin Concentrations

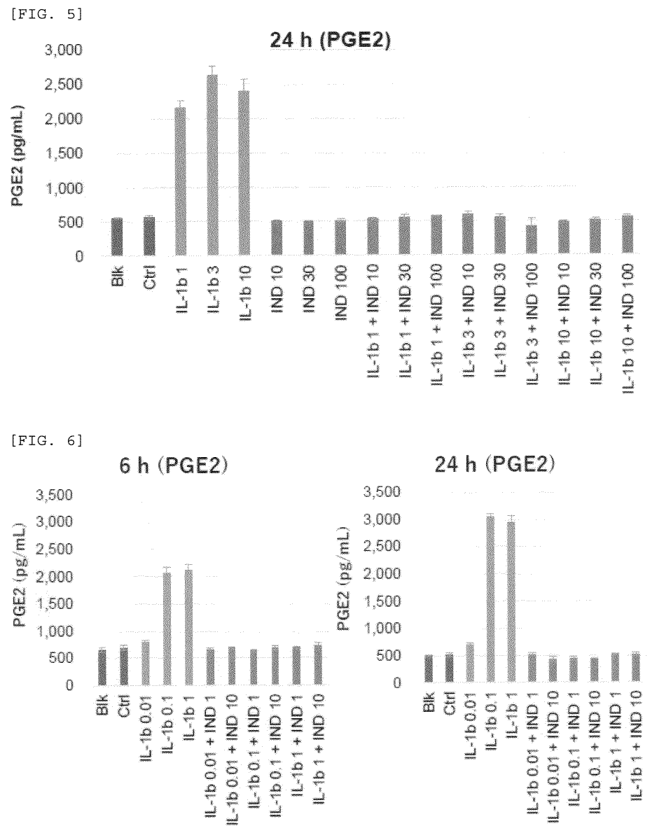

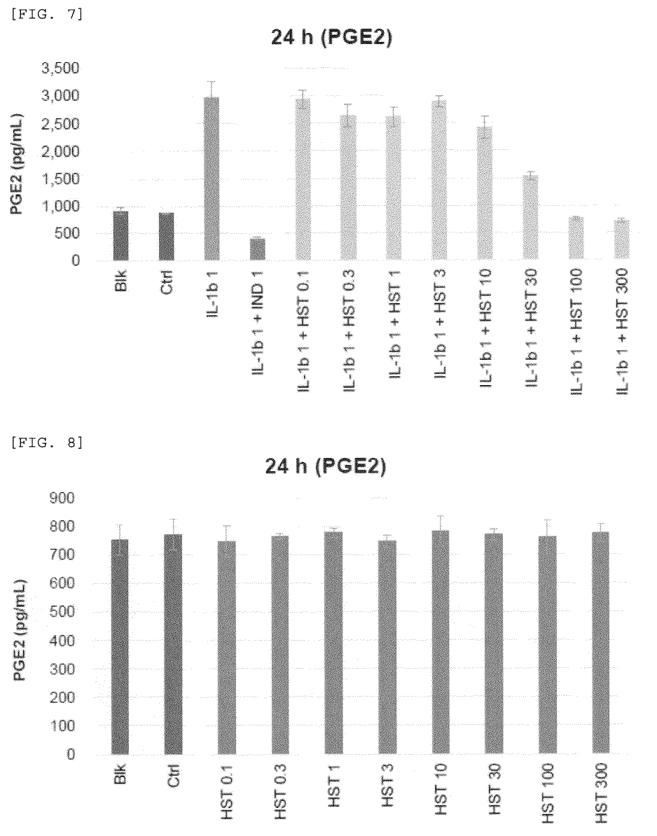

In view of the results in <Examination 1 of Conditions of IL-1β and Indomethacin Concentrations> above, IL-1β and indomethacin at lower concentrations were examined. The examination was conducted under the conditions of a cell count of 4×10⁴ cells/well, IL-1β at 0.01, 0.1, and 1 ng/mL, indomethacin at 1 and 10 UM, and the treatment periods of 6 and 24 h. With the treatment periods of both 6 and 24 h, PGE2 production was caused by IL-1β at 0.1 and 1 ng/ml, but PGE2 was not produced at 0.01 ng/ml (FIG. 6). On the other hand, regarding the action of indomethacin on IL-1β, the PGE2 production was inhibited at both 1 and 10 μM (FIG. 6). Here, no remarkable cytotoxicity was observed under any of the conditions (not shown in the figures).

From the above results, the assay condition was determined to be IL-1β at 1 ng/mL and indomethacin at 1 μM. It was determined that the treatment period of 24 h, with which the PGE2 production due to IL-1β was remarkable and the inhibitory action was more easily detected, was appropriate.

### Example 1

### Construction of Bioassay System:

### <Examination of Condition of Hangeshashinto Extract Concentration>

Using a hangeshashinto extract (hereinafter, referred to “HST”) as a test sample, the concentration exhibiting an inhibitory action on PGE2 production was examined in the same manner as in Reference Example 1. The cell count was 4×10⁴ cells/well, and IL-1β was at 1 ng/ml. Indomethacin as a control was 1 μM, and the treatment periods were 6 and 24 h. The HST concentrations used for the examination were 0.1, 0.3, 1, 3, 10, 30, 100, and 300 g/mL. HST was suspended at a concentration of 15 mg/mL using a serum-free culture medium for human oral keratinocytes (Oral Keratinocyte Medium: OKM, ScienCell, Cat #2611), appropriately stirred and subjected to centrifugation or the like, and the obtained HST was appropriately diluted with a serum-free culture medium and used. With the treatment period of 6 h, the inhibition of PGE2 production started with HST at around 10 μg/mL, and the PGE2 production was inhibited in a concentration dependent manner until 300 μg/mL (not shown in the figures). Similarly, with the treatment period of 24 h, the inhibition of PGE2 production also started at around 10 μg/mL, and the PGE2 production was inhibited in a concentration dependent manner until 300 μg/mL (FIG. 7). At 100 and 300 μg/mL, the PGE2 production was inhibited to the same extent to that of indomethacin (FIG. 7). The IC₅₀ of HST with the treatment period of 24 h was 31.63 μg/mL. No cytotoxicity was observed under any of the conditions in the assay (not shown in the figures).

Moreover, it was found that PGE2 was not produced with the treatment with the HST extract alone (FIG. 8). Furthermore, regarding the cytotoxicity of the treatment with the HST extract alone, it was also found that there was no remarkable influence on the cytotoxicity with the concentration up to 300 μg/mL and the treatment period up to 24 h (not shown in the figures).

From the above examination results, it was found that bioassay of pharmacological activity of hangeshashinto can be conducted by adding indomethacin to human oral keratinocytes, then adding hangeshashinto (HST), culturing the human oral keratinocytes and then measuring the prostaglandin E2 concentration of the culture supernatant. Moreover, it was also found that a preferable assay condition for the inhibitory action of HTS on PGE2 production is a cell count of 4×10⁴ cells/well, IL-1β at 1 ng/mL, indomethacin as a positive control at 1 μM, a culture period of 24 h, and HST at 30 μg/mL (around IC₅₀).

### Example 2

### Use of Bioassay System:

### <Comparison of Difference in Inhibitory Action of Hangeshashinto Extract on PGE2 Production Among Lots>

Using the assay condition determined in Example 1 (a cell count of 4×10⁴ cells/well, IL-18 at 1 ng/ml, indomethacin as a positive control at 1 μM, a culture period of 24 h, and HST at 30 μg/mL (around IC₅₀)), 101 lots of the hangeshashinto extract were measured. To verify that the assays were conducted appropriately in all the experiments, indomethacin at 1 μM was set as a positive control. Moreover, because it was not possible to conduct assays of the 101 lots of HST on the same day on the same plate, a representative lot (Lot 392107100) was set as an internal control to correct the errors among the assays.

It was found that all the 101 lots of HST inhibited the PGE2 production caused by IL-1β from 16 assays in total (not shown in the figures).

From the above measurement, the inhibition ratio of each treatment was calculated based on the PGE2 production amount with IL-1β regarded as 100%, and the values in a same plate were corrected using the average value of the representative lot. Furthermore, the values of the different plates were corrected using the average values of the all representative lot.

$$\{ ( Measurement Value / Average Value of ES 0 Measurement Values of Same Plate ) \times 100 / Average Value of Inhibition Ratios of Representative Lot on Same Plate \} \times Average Value of Inhibition Ratios of Representative Lot of All Plates \qquad [ Math . 1 ]$$

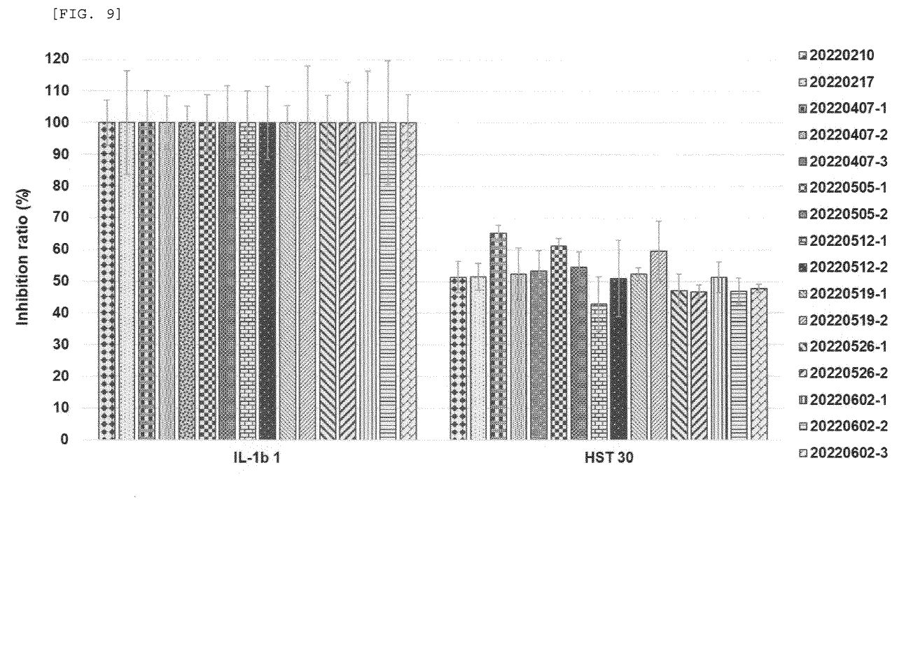

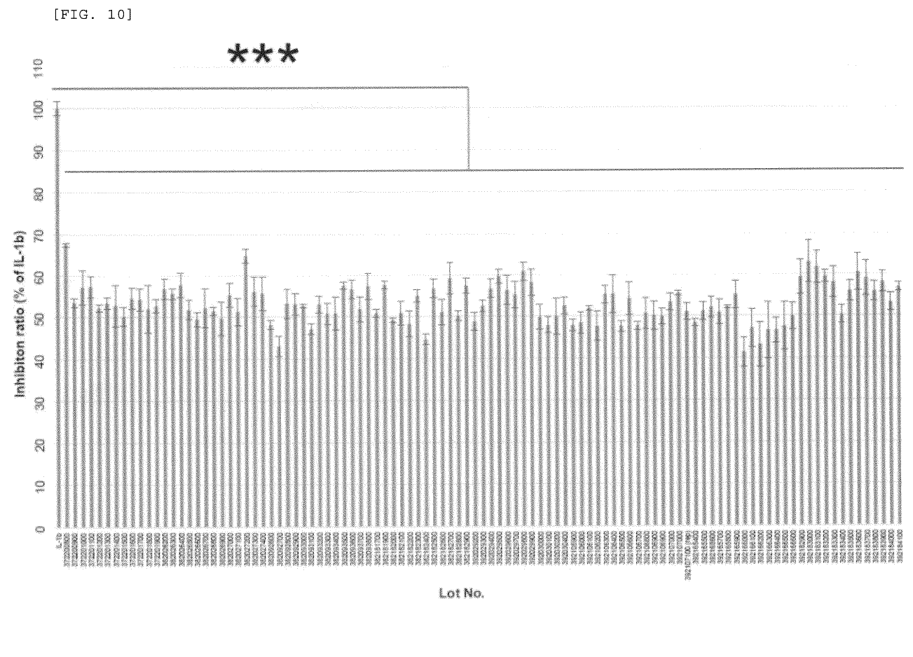

The experimental errors of this test were examined from a repeating test using the HST extract of the representative lot (30 μg/mL). The errors among the dates were calculated from the results of all the plates of the 16 assays in total, and the errors among the plates were calculated from the results of two or three plates in assays on a same day. As a result, the errors among the dates of this test were about CV=11.06% (calculated from the 16 assays in total), and the errors among the plates were CV=0.80 to 12.52% (calculated from plates of assays on a same day (six days in total)) (FIG. 9).

Compared to the results of the treatment with IL-1β alone, all the lots of HST inhibited the PGE2 production caused by IL-1β with significance (FIG. 10). Moreover, the PGE2 production ratios of the lots of HST were in the range of 41.33 to 67.38%, and the maximum difference among the lots was about 25% (the most potent pharmacological action was a PGE2 production inhibition ratio of 58.67% of Lot No. 392166000 (sample No. 75) while the weakest pharmacological action was a PGE2 production inhibition ratio of 32.62% of Lot No. 372200800 (sample No. 103)). The average value of the PGE2 production ratios of the 101 lots was 53.06±0.46% (SEM). Here, the PGE2 production ratios of the hangeshashinto extract lots based on the increase in PGE2 caused by the treatment with IL-1β in FIG. 10 were analyzed by one-way ANOVA and Dunnett's test.

From the above results, it was found that all the HST lots measured had an inhibitory action on PGE2 production although the there was a difference in the potency of the action. It was also found that the values of different lots can be corrected using a positive control.

The bioassay method for pharmacological activity of hangeshashinto of the invention can be used for controlling the quality of hangeshashinto.

1. A bioassay method for pharmacological activity of hangeshashinto, comprising:
adding an inflammatory cytokine and a hangeshashinto-containing test sample to a human oral keratinocyte;
culturing the human oral keratinocyte; and
then measuring the prostaglandin E2 concentration of the culture supernatant.

2. The bioassay method for pharmacological activity of hangeshashinto according to claim 1, wherein the inflammatory cytokine is interleukin-1β.

3. The bioassay method for pharmacological activity of hangeshashinto according to claim 1, wherein a nonsteroidal anti-inflammatory agent is further used as a positive control.

4. The bioassay method for pharmacological activity of hangeshashinto according to claim 3, wherein the nonsteroidal anti-inflammatory agent is indomethacin or diclofenac sodium.

5. The bioassay method for pharmacological activity of hangeshashinto according to claim 1, wherein the culturing is conducted for 4 to 36 hours.

6. A quality control method of hangeshashinto, comprising the following (a), (b), and (d):
(a) a step of adding an inflammatory cytokine to a human oral keratinocyte, culturing the human oral keratinocyte, and then measuring the prostaglandin E2 concentration of the culture supernatant;
(b) a step of adding the inflammatory cytokine and a hangeshashinto-containing test sample to a human oral keratinocyte, culturing the human oral keratinocyte, and then measuring the prostaglandin E2 concentration of the culture supernatant; and
(d) comparing the prostaglandin E2 concentrations measured in (a) and (b).

7. A quality control method of hangeshashinto, comprising the following (a) to (c) and (e):
(a) adding an inflammatory cytokine to a human oral keratinocyte, culturing the human oral keratinocyte, and then measuring the prostaglandin E2 concentration of the culture supernatant;
(b) adding the inflammatory cytokine and a hangeshashinto-containing test sample to a human oral keratinocyte, culturing the human oral keratinocyte, and then measuring the prostaglandin E2 concentration of the culture supernatant;
(c) adding the inflammatory cytokine and a nonsteroidal anti-inflammatory agent as a positive control to a human oral keratinocyte, culturing the human oral keratinocyte, and then measuring the prostaglandin E2 concentration of the culture supernatant; and
(e) comparing the prostaglandin E2 concentrations measured in (a) to (c).

8. The quality control method of hangeshashinto according to claim 6, wherein the inflammatory cytokine is interleukin-1β.

9. The quality control method of hangeshashinto according to claim 7, wherein the nonsteroidal anti-inflammatory agent is indomethacin or diclofenac sodium.

10. The quality control method of hangeshashinto according to claim 6, wherein the culturing is conducted for 4 to 36 hours.

11. The quality control method of hangeshashinto according to claim 7, wherein the prostaglandin E2 concentration measured in (c) is compared with the prostaglandin E2 concentration of another lot to correct the values of lots.

12. The quality control method of hangeshashinto according to claim 7, wherein the inflammatory cytokine is interleukin-1β.

13. The quality control method of hangeshashinto according to claim 7, wherein the culturing is conducted for 4 to 36 hours.
